(19) United States
(12) Patent Application Publication
(10) Pub. No.: US 2024/0057902 A1
(43) Pub. Date: Feb. 22, 2024

(54) APPARATUS AND METHOD FOR MEASURING BLOOD COMPONENTS

(71) Applicants: SAMSUNG ELECTRONICS CO., LTD., Suwon-si (KR); Korea University Research and Business Foundation, Seoul (KR)

(72) Inventors: Yun S PARK, Suwon-si (KR); Seoung Bum KIM, Seoul (KR); Chunghyup MOK, Seoul (KR); Jinsoo BAE, Seoul (KR); Leekyung YOO, Seongnam-si (KR); Yeol Ho LEE, Suwon-si (KR); Joon Hyung LEE, Suwon-si (KR); Keewon JEONG, Busan (KR); Yoon Sang CHO, Gongju-si (KR)

(73) Assignees: SAMSUNG ELECTRONICS CO., LTD., Suwon-si (KR); Korea University Research and Business Foundation, Seoul (KR)

(21) Appl. No.: 18/106,825

(22) Filed: Feb. 7, 2023

(30) Foreign Application Priority Data
Aug. 17, 2022 (KR) 10-2022-0102763

(51) Int. Cl. A61B 5/145; A61B 5/0537; A61B 5/00; G16H 10/40; G16H 10/60

(52) U.S. Cl. CPC A61B 5/14546; A61B 5/0537; A61B 5/7267; A61B 5/7246; G16H 10/40; G16H 10/60; A61B 2560/0223

(57) ABSTRACT

An apparatus for measuring blood components, includes: an impedance sensor configured to measure a bio-impedance of a user; and a processor configured to measure, by using a multiple-output artificial neural network (ANN) learning model, a concentration of a basic blood component and a concentration of at least one auxiliary blood component associated with the basic blood component, based on user metadata and the measured bio-impedance.

### CROSS-REFERENCE TO RELATED APPLICATION(S)

This application claims priority from Korean Patent Application No. 10-2022-0102763, filed on Aug. 17, 2022, in the Korean Intellectual Property Office, the disclosure of which is incorporated by reference herein in its entirety.

### BACKGROUND

### 1. Field

Example embodiments of the disclosure relate non-invasively measuring blood components by using bio-impedance.

### 2. Description of Related Art

Research on information technology (IT)-medical convergence technology, in which IT and medical technology are combined, is being conducted to address a variety of difficult medical related issues including the aging population structure, rapid increase in medical expenses, and shortage of specialized medical service personnel. Accordingly, the monitoring of health conditions associated with the human body is no longer limited to fixed places, such as a hospital, but is expanding to include a mobile healthcare sector established for monitoring a user's health condition at any time and any place in normal daily life.

### SUMMARY

According to an aspect of an example embodiment, an apparatus for measuring blood components, includes: an impedance sensor configured to measure a bio-impedance of a user; and a processor configured to measure, by using a multiple-output artificial neural network (ANN) learning model, a concentration of a basic blood component and a concentration of at least one auxiliary blood component associated with the basic blood component, based on user metadata and the measured bio-impedance.

The multiple-output ANN learning model is pre-trained to output the concentration of the basic blood component and the concentration of the at least one auxiliary blood component.

The processor may be further configured to determine, as the at least one auxiliary blood component, at least one blood component that has a correlation with the basic blood component that is greater than or equal to a threshold value.

The processor may be further configured to, when outputting the basic blood component and the at least one blood component, guide remeasurement based on the correlation between the basic blood component and the at least one auxiliary blood component falling outside a correlation range.

The basic blood component includes triglyceride, and the at least one auxiliary blood component includes uric acid and creatinine.

The processor may be further configured to obtain the user metadata via a user interface, and the user metadata includes age, gender, height, and weight.

The processor may be further configured to, based on the measured concentration of the basic blood component and the measured concentration of the at least one auxiliary blood component, provide the user with health guidance, and the health guidance includes at least one of a warning, diet recommendations, or exercise information.

The multiple-output ANN learning model includes an input layer, a plurality of hidden layers, and an output layer, and the plurality of hidden layers include at least one of a linear function, a batch normalization function, a rectified linear unit function, and a dropout function.

The processor may be further configured to perform preprocessing on the measured bio-impedance and the user metadata by standard scaling.

The processor may be further configured to obtain an impedance index based on the measured bio-impedance and the user metadata, and input the impedance index into the multiple-output ANN learning model.

The impedance index may include a value obtained by dividing the user metadata by the measured bio-impedance.

According to an aspect of an example embodiment, a method of measuring blood components by an apparatus for measuring blood components, includes: measuring, by an impedance sensor, a bio-impedance of a user; obtaining user metadata from the user through a user interface, the user metadata including at least one of age, gender, height, and weight; and measuring, by using a multiple-output artificial neural network (ANN) learning model, a concentration of a basic blood component and a concentration of at least one auxiliary blood component associated with the basic blood component, based on the measured bio-impedance and the user metadata.

The multiple-output ANN learning model may be pre-trained to output the concentration of the basic blood component and the concentration of the at least one auxiliary blood component.

The at least one auxiliary blood component may include a blood component having a correlation with the basic blood component that may be greater than or equal to a predetermined threshold value.

The method may further include, based on the measured concentration of the basic blood component and the measured concentration of the at least one auxiliary blood component, providing the user with health guidance including at least one of a warning, diet recommendations, and exercise information.

The method may further include performing preprocessing on the measured bio-impedance and the user metadata by standard scaling.

The measuring the concentration of the basic blood component and the concentration of the at least one auxiliary blood component may include: generating an impedance index based on the measured bio-impedance and the user metadata, and inputting the impedance index into the multiple-output ANN learning model.

According to an aspect of an example embodiment, an electronic device includes: a memory storing one or more instructions; and a processor configured to execute the one or more instructions to: input a bio-impedance of a user and user metadata into a multiple-output artificial neural network (ANN) learning model, and output a concentration of a basic blood component and a concentration of at least one auxiliary blood component associated with the basic blood component.

The multiple-output ANN learning model may be pre-trained to output the concentration of the basic blood component and the concentration of the at least one auxiliary blood component.

The processor may be further configured to execute the one or more instructions determine, as the at least one auxiliary blood component, at least one blood component having a correlation with the basic blood component that is greater than or equal to a threshold value.

### BRIEF DESCRIPTION OF THE DRAWINGS

The above and other aspects, features, and advantages of certain embodiments of the present disclosure will be more apparent from the following description taken in conjunction with the accompanying drawings, in which:

FIG. 1 is a block diagram illustrating an apparatus for measuring blood components according to an embodiment;

FIG. 2A is a diagram illustrating an example of estimating blood components using a single-output artificial neural network (ANN) learning model;

FIG. 2B is a diagram illustrating an example of estimating blood components using a multiple-output artificial neural network (ANN) learning model;

FIG. 3 is a block diagram illustrating a multiple-output artificial neural network (ANN) learning model according to an embodiment;

FIGS. 4A and 4B are block diagrams illustrating a multiple-output artificial neural network (ANN) learning model according to an embodiment;

FIG. 5 is a diagram illustrating a correlation coefficient heatmap between blood components;

FIG. 6 is a block diagram illustrating an apparatus for measuring blood components according to another embodiment of the present disclosure;

FIGS. 7A and 7B are diagrams illustrating an example of providing health guidance information which is performed by a processor of FIGS. 1 and 6;

FIG. 8 is a flowchart illustrating a method of measuring blood components according to an embodiment; and

FIGS. 9 to 11 are diagrams illustrating examples of structures of an electronic device including the aforementioned apparatus for measuring blood components.

### DETAILED DESCRIPTION

Details of other embodiments are included in the following detailed description and drawings. Advantages and features of the present invention, and a method of achieving the same will be more clearly understood from the following embodiments described in detail with reference to the accompanying drawings. Throughout the drawings and the detailed description, unless otherwise described, the same drawing reference numerals will be understood to refer to the same elements, features, and structures. The relative size and depiction of these elements in the drawings may be exaggerated for clarity, illustration, and convenience.

It will be understood that, although the terms first, second, etc. may be used herein to describe various elements, these elements should not be limited by these terms. These terms are only used to distinguish one element from another. Any references to singular may include plural unless expressly stated otherwise. In addition, unless explicitly described to the contrary, an expression such as “comprising” or “including” will be understood to imply the inclusion of stated elements but not the exclusion of any other elements. Also, the terms, such as “unit” or “module”, etc., should be understood as a unit for performing at least one function or operation and that may be embodied as hardware, software, or a combination thereof.

As used herein, expressions such as “at least one of,” when preceding a list of elements, modify the entire list of elements and do not modify the individual elements of the list. For example, the expression, “at least one of a, b, and c,” should be understood as including only a, only b, only c, both a and b, both a and c, both b and c, or all of a, b, and c.

Hereinafter, various embodiments of an apparatus and method for measuring blood components will be described with reference to the accompanying drawings. Various embodiments thereof may be included in an electronic device, such as a smartphone, a tablet PC, a desktop computer, a laptop computer, or a wearable electronic device such as a wristwatch wearable device, a bracelet wearable device, a wristband wearable device, a ring wearable device, a glasses wearable device, an earphone wearable device, a necklace wearable device, an anklet wearable device, and a headband wearable device.

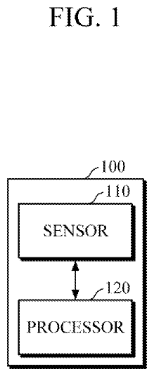

FIG. 1 is a block diagram illustrating an apparatus for measuring blood components according to an embodiment of the present disclosure.

Referring to FIG. 1, an apparatus 100 for measuring blood components includes a sensor 110 and a processor 120.

The sensor 110 may include an impedance sensor for measuring bio-impedance of a user. For example, the impedance sensor may include a pair of current electrodes and a pair of voltage electrodes configured to measure impedance by a four-electrode method. When a user's skin is in contact with the current electrodes and the voltage electrodes, impedance may be measured by applying a current to the current electrodes and measuring a voltage using the pair of voltage electrodes. Alternatively, impedance may be measured by applying a constant voltage to the pair of voltage electrodes and measuring a current flowing through the pair of current electrodes. However, the impedance measurement is not limited thereto. For example, the impedance sensor may be configured to measure impedance by using a two-electrode method. The sensor 110 may further include, for example, a Photoplethysmogram (PPG) sensor, an Electrocardiography (ECG) sensor, an Electromyography (EMG) sensor, etc., and may be formed as a single chip.

The processor 120 may be connected to the sensor 110 and be configured to control the sensor 110. The processor 120 may receive data from the sensor 110 and measure the concentration of a blood component, such as triglyceride, uric acid, creatinine, blood carotenoid, glucose, lactate, total protein, cholesterol, ethanol, but is not limited thereto.

For example, the processor 120 may measure the concentration of a blood component by using a machine learning model based on the bio-impedance (e.g., measured by the impedance sensor) and metadata regarding a user. In particular, the processor 120 may measure concentrations of a plurality of blood components by using a multiple-output artificial neural network (ANN) learning model. The user metadata may include at least one of a user's age, gender, height, and weight, and the processor 120 may collect the metadata, for example, from the user through a user interface.

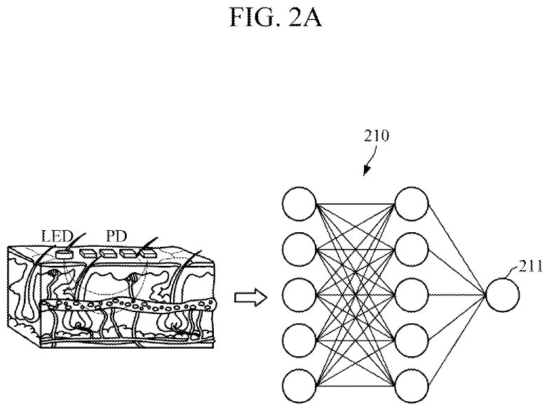

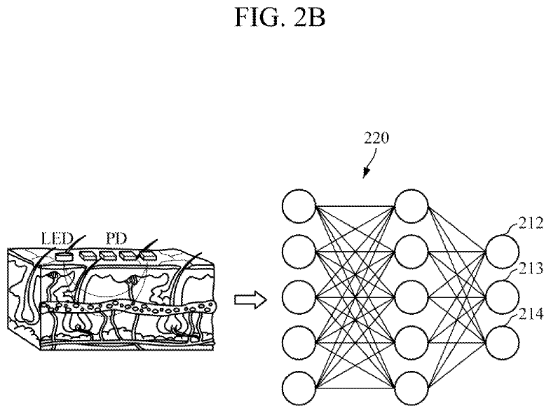

FIG. 2A is a diagram illustrating an example of estimating blood components using a single-output artificial neural network (ANN) learning model, and FIG. 2B is a diagram illustrating an example of estimating blood components using a multiple-output artificial neural network (ANN) learning model.

In the case where bio-signals are acquired using, for example, a light source light emitting diode (LED) and a detector photodiode (PD), and blood components are output by each of a single-output ANN learning model 210 and a multiple-output ANN learning model 220 which are trained by learning the same amount of acquired bio-signals, the multiple-output ANN learning model 220 is trained more than the single-output ANN learning model 210, such that blood components 212, 213, and 214, having high correlation with each other and output by the multiple-output ANN learning model 220, may be estimated relatively accurately compared to a blood component 211 output by the single-output ANN learning model 210. In this case, by using the multiple-output ANN learning model 220, not only the blood component 212 to be finally obtained, but also the blood components 213 and 214 having a high correlation may be measured together.

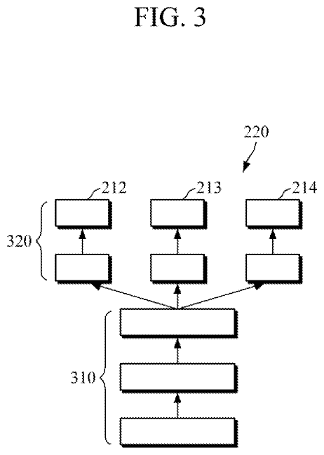

FIG. 3 is a block diagram illustrating a multiple-output artificial neural network (ANN) learning model according to an embodiment of the present disclosure.

Referring to FIG. 3, the multiple-output ANN learning model 220 may be trained to output a plurality of output values 212, 213, and 214. In this case, the multiple-output ANN learning model 220 may include a task specific layer 320 and a shared layer 310, in which by learning parameters of the task specific layer 320 and the shared layer 310, the plurality of output values 212, 213, and 214 may be output according to input values. In this case, the shared layer 310 may include a common parameter, such that by simultaneously learning the correlation between multiple blood components, the performance of the model may be improved.

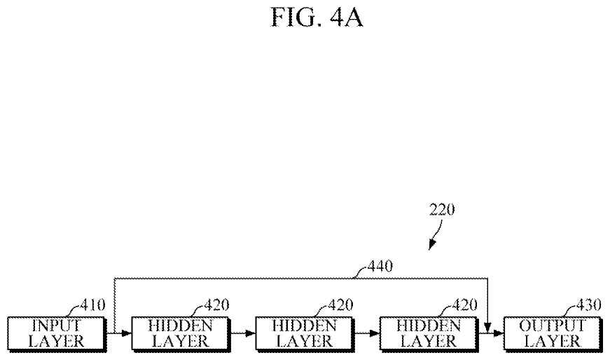

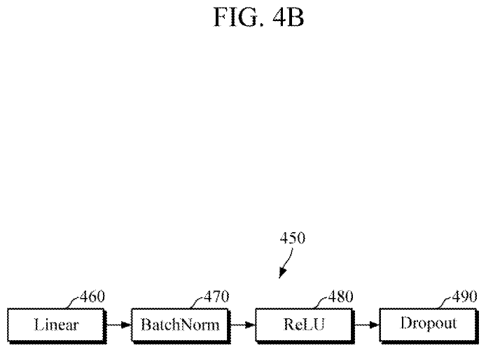

FIGS. 4A and 4B are block diagrams illustrating a multiple-output artificial neural network (ANN) learning model according to another embodiment of the present disclosure.

Referring to FIG. 4A, the multiple-output ANN learning model 220 may include an input layer 410, a plurality of hidden layers 420, and an output layer 430.

The input layer 410 may include, for example, 126 nodes by considering impedance obtained from a user, and user metadata, and the output layer 430 may include, for example, three nodes by considering the number of blood components to be output.

The plurality of hidden layers 420 may include two or more multi-layer perceptron (MLP) blocks configured to extract features of a desired blood component (e.g., triglyceride), in which the number of MLP blocks may be set to, for example, three by considering the number of impedance data and in order to prevent overfitting during training. In this case, the number of nodes in the respective MLP blocks may be set to, for example, 63 which is half of the 126 nodes set in the input layer 410 for extracting key features in the nodes.

In addition, referring to FIG. 4B, an MLP block 450 of the hidden layers 420 may include, for example, at least one of a linear function 460, a batch normalization (BatchNorm) function 470, a rectified linear unit (ReLU) function 480, and a dropout function 490.

Here, the BatchNorm function performs normalization for each batch to overcome hindrance to training which may occur due to a difference in units between impedance sensors. The ReLU function may be a function that performs the smallest amount of computation among nonlinear conversion functions. The Dropout function may be a function for preventing overfitting which may occur due to a relatively small number of data.

Referring back to FIG. 4A, in order to prevent the problem of overfitting which occurs as the learning model increases in depth, residual connection may be performed between the input layer 410 and the output layer 430. However, the structure of the multiple-output ANN learning model 220 is not limited thereto.

Referring back to FIG. 1, the processor 120 may be configured to measure concentrations of the plurality of blood components by using the multiple-output ANN learning model based on the measured bio-impedance and the user metadata.

In this case, the plurality of blood components may include a basic blood component and one or more auxiliary blood components associated with the basic blood component, and the multiple-output ANN learning model may be a multiple-output ANN learning model pre-trained to output concentrations of the basic blood component and the one or more auxiliary blood components.

The processor 120 may be configured to first determine, as the auxiliary blood component, a blood component having a correlation with the basic blood component which is a final blood component to be obtained, the correlation being greater than or equal to a predetermined threshold value.

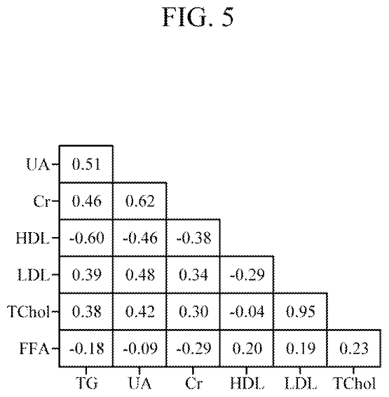

FIG. 5 is a diagram illustrating a correlation coefficient heatmap between blood components. In FIG. 5, the horizontal axis and vertical axis represent triglyceride TG, uric acid UA, creatinine Cr, high density lipoprotein cholesterol HDL, low-density lipoprotein cholesterol LDL, total cholesterol TChol, and free fatty acid FFA, and the numbers indicate correlation coefficients. For example, assuming that the basic blood component to be obtained is triglyceride, and a predetermined correlation coefficient threshold is 0.4, blood components having a correlation coefficient of greater than or equal to 0.4 with respect to triglyceride are creatinine (correlation coefficient: 0.46) and uric acid (correlation coefficient: 0.51), such that creatinine and uric acid may be determined as the auxiliary blood components. In this case, the correlation coefficient threshold, the basic blood component, and the auxiliary blood component are not limited thereto.

The processor 120 may perform preprocessing on the measured bio-impedance and the user metadata. For example, the processor 120 may preprocess the measured bio-impedance and the user metadata by standard scaling in which a mean value of feature values is changed to 0 and variance is changed to 1, so that all the feature values have the same scale. However, the preprocessing method is not limited thereto.

The processor 120 may input the preprocessed bio-impedance and metadata of the user to the multiple-output ANN learning model 220. In this case, the processor 120 may obtain an impedance index based on the measured bio-impedance and the user metadata, and may input the obtained impedance index to the multiple-output ANN learning model 220.

For example, the processor 120 may obtain the impedance index based on the bio-impedance and the user metadata which are obtained for each frequency according to body parts (e.g., arm, leg, torso, etc.). For example, the processor 120 may obtain, as the impedance index, a value obtained by dividing the user metadata by the measured bio-impedance (e.g., the square of height/impedance). However, the impedance index is not limited thereto.

The processor 120 may measure a concentration of a predetermined basic blood component and concentrations of auxiliary blood components, having a correlation with the basic blood component that is greater than or equal to a threshold value, as output values of the multiple-output ANN learning model 220.

In this case, the processor 120 may output only the basic blood component, which is finally desired by a user, among the measured concentrations of the plurality of blood components through, for example, a display or may also simultaneously output all the concentrations of the basic blood component and the auxiliary blood components.

By calibrating the concentration of the basic blood component based on the measured concentrations of the auxiliary blood components, the processor 120 may measure the concentration of the basic blood component more accurately. For example, assuming that the basic blood component is triglyceride, and the plurality of auxiliary blood components are uric acid and creatinine, the processor 120 may measure a final triglyceride concentration by calibrating a triglyceride concentration based on concentrations of uric acid and creatinine by using a predetermined calibration method (e.g., linear regression analysis).

Further, while outputting the basic blood component and the auxiliary blood components, the processor 120 may guide remeasurement if a correlation between the basic blood component and the auxiliary blood component falls outside a predetermined correlation range. For example, if a correlation between the basic blood component (e.g., triglyceride) and the auxiliary blood component (e.g., creatinine) falls outside a predetermined correlation range (e.g., between 0.3 and 0.6), the processor 120 may determine that there is an error in the measured correlation and may guide remeasurement.

The processor 120 may provide a user with health guidance including at least one of a warning, diet recommendations, and exercise information based on the measured concentration of the blood component.

For example, upon measuring a triglyceride level, the processor 120 may generate user-customized health guidance information, such as a warning, diet recommendations, and exercise information, based on the measured triglyceride level, and may provide the information to the user through various output means. For example, the processor 120 may determine whether the measured triglyceride level is normal (e.g., less than 150 mg/dL), borderline (e.g., 150 mg/dL to 199 mg/dL), or high (e.g., 200 mg/dL or more). For example, Upon determining that the triglyceride level is normal, the processor 120 may provide the normal level to a user, and may guide the user to maintain the current diets or exercise. Alternatively, upon determining that the triglyceride level is at the borderline (e.g., 150 mg/dL to 199 mg/dL) or high (e.g., 200 mg/dL or more), the processor 120 may provide warning information using, for example an alarm, and/or message, and the processor 120 may provide the user with recommended diet or exercise which may be commonly applied.

The processor 120 may collect health data, such as a user's diet data (e.g., ingested food, amount of food intake, and/or number of times of food intake per day), exercise data (activity level, type of exercise performed, and/or amount of exercise per day), blood pressure, body mass index (BMI) score, underlying medical conditions, and/or previous measured triglyceride level, through the user interface or from a healthcare application installed in another electronic device. The processor 120 may analyze the collected user metadata, the user's diet information, exercise information, and/or health data, etc., and may provide the user with guidance, such as customized diet or exercise, based on the analysis. For example, even when the current measured triglyceride level falls within the normal range, if there are factors that adversely affect the triglyceride level in the user's current diet data, exercise data, and/or the health data, the processor 120 may guide the user to remove or reduce the factors. Further, if the triglyceride level is at the borderline level (e.g., 150 mg/dL to 199 mg/dL) or high level (e.g., 200 mg/dL or more), the processor 120 may provide guidance by generating recommendations of a customized diet or exercise plan for the user.

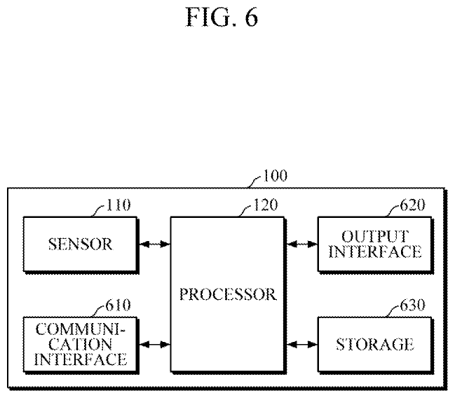

FIG. 6 is a block diagram illustrating an apparatus for measuring blood components according to another embodiment of the present disclosure.

Referring to FIG. 6, the apparatus 100 for measuring blood components may include the sensor 110, the processor 120, a communication interface 610, an output interface 620, and a storage 630. The sensor 110 and the processor 120 are described in detail above, such that a description thereof will be omitted.

The communication interface 610 may be configured to communicate with another electronic device under the control of the processor 120 by using communication techniques. The communication interface 610 may transmit sensor data measured by the sensor 110 and blood component data generated and processed by the processor 120 to the electronic device. By using an installed healthcare application, the electronic device may manage the blood component data received from the apparatus 100 for measuring blood components, body composition information (e.g., skeletal muscle mass, basal metabolic rate, body water, body fat percentage) and/or exercise information (e.g., step count, running distance) and may provide the data to a user. The communication interface 610 may receive user information and data (e.g., a user's health data, diet data, exercise data) from the electronic device. The communication interface 610 may receive a learning model generated by the electronic device, or may receive a user's bio-impedance measured by an impedance sensor of the electronic device. In this case, the processor 120 may measure a user's blood component by using the bio-impedance received from the electronic device through the communication interface 610.

As used in this disclosure, the electronic communication techniques may include Bluetooth communication, Bluetooth Low Energy (BLE) communication, Near Field Communication (NFC), WLAN communication, Zigbee communication, Infrared Data Association (IrDA) communication, Wi-Fi Direct (WFD) communication, Ultra-Wideband (UWB) communication, Ant+ communication, WIFI communication, mobile communication, but are not limited thereto.

The output interface 620 may output the sensor data measured by the sensor 110, the data generated and processed by the processor 120, and/or the data received through the communication interface 610. For example, the output interface 620 may output a user interface to a display so that a user may input a variety of information. Alternatively, the output interface 620 may output guidance information, including the measured concentrations of the blood components, a warning, diet recommendations, and exercise information, which are generated by the processor 120, by using, for example, a display, a speaker, and/or a haptic device.

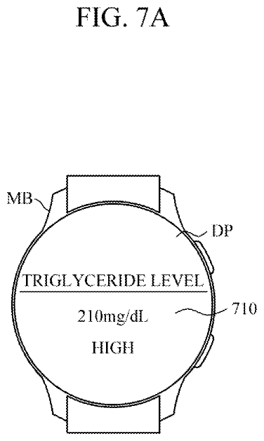

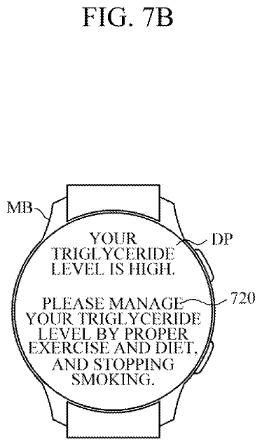

FIGS. 7A and 7B are diagrams illustrating an example of providing health guidance information which is performed by the processor 120 of FIGS. 1 and 6. However, the following examples are provided merely for convenience of explanation, and the present disclosure is not limited thereto.

Referring to FIG. 7A, the processor 120 may output, through the output interface 620, the concentration of a blood component (e.g., triglyceride) and/or graphic objects (e.g., text, icons, images), indicating whether the blood component concentration is normal, to a display DP of a main body MB. In this case, based on whether the blood component concentration is normal, the processor 120 may output the graphic objects in different types and colors, so that the user may easily distinguish between normal and abnormal concentrations. For example, normal concentrations may be in green while abnormal concentrations may be in red.

Referring to FIG. 7B, if the measured triglyceride level is abnormal as illustrated herein, the processor 120 may output a warning text 720, such as “your triglyceride level is high. Please manage your triglyceride level by proper exercise and diet, and stopping smoking.”

Referring back to FIG. 6, the storage 630 may store various instructions to be executed by the processor 120. In addition, the storage 630 may store data generated and/or processed by the sensor 110, the processor 120, the communication interface 610, etc., which may be referred to by the processor 120 during measurement of blood components. For example, the storage 630 may store health guidance information, such as user information, a user's diet data, exercise data, and health data, recommended diet and exercise, etc., learning model, blood component calibration model, and the like.

The storage 630 may include at least one non-transitory storage medium such as flash memory, a hard disk, a multimedia card micro memory, a card memory (e.g., an SD memory, an XD memory), a Random Access Memory (RAM), a Static Random Access Memory (SRAM), a Read Only Memory (ROM), an Electrically Erasable Programmable Read Only Memory (EEPROM), a Programmable Read Only Memory (PROM), a magnetic memory, a magnetic disk, and an optical disk, but is not limited thereto.

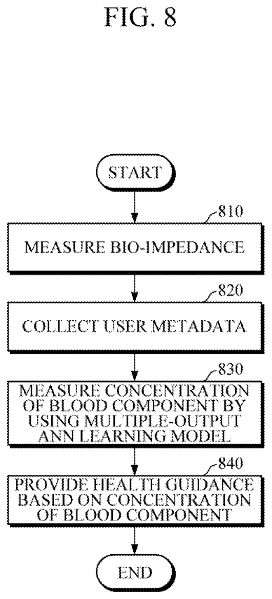

FIG. 8 is a flowchart illustrating a method of measuring blood components according to an embodiment of the present disclosure.

The method of FIG. 8 is an example of a method for measuring blood components performed by the apparatus 100, which is described in detail above, and thus will be briefly described below.

First, the apparatus 100 for measuring blood components may measure a user's bio-impedance by using an impedance sensor 110 (operation 810).

The apparatus for measuring blood components may collect user metadata, including, for example, age, gender, height, and/or weight, from a user through a user interface (operation 820). As described above, the user metadata may include at least one of a user's age, gender, height, and weight, but is not limited thereto.

The apparatus 100 for measuring blood components may measure concentrations of a plurality of blood components by using a multiple-output ANN learning model 220 based on the measured bio-impedance and the user metadata (operation 830). In this case, the plurality of blood components may include a basic blood component and one or more auxiliary blood components associated with the basic blood component, and the multiple-output ANN learning model 220 may be an ANN learning model pre-trained to output a concentration of the basic blood component and concentrations of the one or more auxiliary blood components. For example, the auxiliary blood component may have a correlation with the basic blood component that is greater than or equal to a predetermined threshold value.

The apparatus 100 for measuring blood components may obtain an impedance index based on the measured bio-impedance and the user metadata, and may output the plurality of blood components by inputting the obtained impedance index to the multiple-output ANN learning model 220.

The apparatus 100 for measuring blood components may provide health guidance, including at least one of a warning, diet recommendations, and exercise information, to a user based on the measured concentrations of the blood components (operation 840). For example, upon measuring a triglyceride level, the apparatus for measuring blood components may generate user-customized health guidance information, such as a warning, diet recommendations, and exercise information, based on the measured triglyceride level and may provide the information to the user by using various output means.

The apparatus 100 for measuring blood components may preprocess the measured bio-impedance and the user metadata by applying standard scaling.

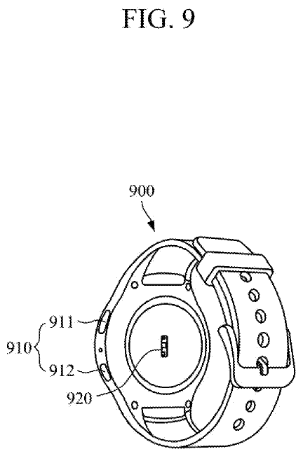

FIGS. 9, 10, and 11 are diagrams illustrating examples of structures of an electronic device including the aforementioned apparatus for measuring blood components. However, the present disclosure is not limited to the illustrated examples.

Referring to FIG. 9, the electronic device may be implemented as a wristwatch wearable device 900, and may include a main body and a wrist strap. A display may be provided on a front surface of the main body, and may display various application screens including time information, received message information, and estimated blood components (e.g., triglyceride). As seen in FIG. 9, an impedance sensor 910 may be disposed on a side surface of the main body. The impedance sensor 910 may include a first electrode part 911 and a second electrode part 912 which are spaced apart from each other to come into contact with two fingers respectively of a user. Each of the first electrode part 911 and the second electrode part 912 may include a respective pair of electrodes.

In addition, the wearable device 900 may further include a sensor device 920 including, for example, a PPG sensor and a force sensor. The sensor device 920 may be disposed on a rear surface of the main body of the wearable device 900. When the main body is worn on a user's wrist, the sensor device 920 may measure, for example, a PPG signal and/or a force signal, at an upper part of the wrist. When the user wears the main body on the wrist of one hand and places a finger of the other hand on the impedance sensor 910, the sensor device 920 may measure the, for example, PPG signal at the upper part of the wrist at the same time while the impedance sensor 910 measures bio-impedance.

A processor 120 and various other components may be disposed in a main body case of the wearable device 900, including, for example, a memory storing one or more instructions. The processor 120, which by executing the one or more instructions, may be configured to input a user's bio-impedance and user metadata into a multiple-output ANN learning model 220 to output concentrations of a plurality of blood components. In this case, the plurality of blood components may include a basic blood component (e.g., triglyceride) and one or more auxiliary blood components (e.g., uric acid, creatinine) having a correlation with the basic blood component that is greater than or equal to a predetermined threshold value, and the multiple-output ANN learning model 220 may be an ANN learning model pre-trained to output a concentration of the basic blood component and concentrations of the one or more auxiliary blood components.

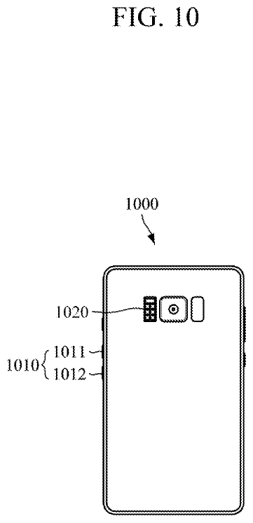

Referring to FIG. 10, the electronic device may be implemented as a mobile device 1000 such as a smartphone.

The mobile device 1000 may include a main body case and a display panel. The main body case may form the exterior of the mobile device 1000. The main body case may have a front surface, on which the display panel and a cover glass may be disposed sequentially, and the display panel may be exposed to the outside through the cover glass. As illustrated in FIG. 10, an impedance sensor 1010, including a first electrode part 1011 and a second electrode part 1012, may be disposed on a side surface of the main body. In addition, a separate sensor device 1020 for measuring, for example, a PPG signal, and a force signal may be disposed on a rear surface of the main body. However, the arrangement is not limited thereto, and the impedance sensor 1010 may be disposed near the sensor device 1020 disposed on the rear surface of the main body; by contrast, the sensor device 1020 may be disposed between the first electrode part 1011 and the second electrode part 1012 of the impedance sensor 1010 or next to the impedance sensor 1010.

A processor 120 and various other components may be disposed in a main body case. The processor 120 may measure blood components by using the bio-impedance measured by the impedance sensor 1010, and when the, for example, PPG signal are measured by the sensor device 1020, the processor may further measure bio-information, such as blood pressure by using the PPG signal.

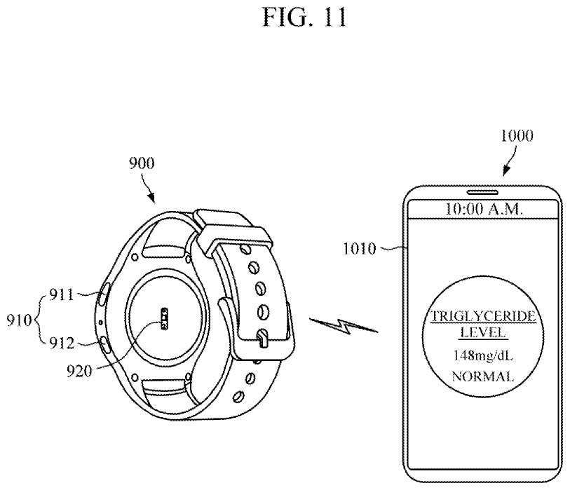

FIG. 11 is a diagram illustrating an example of measuring blood components and providing health guidance by connecting the wearable device 900 with the mobile device 1000. For example, a triglyceride level may be estimated by a processor and the impedance sensor 910 of the wearable device 900, and the mobile device 1000 may receive health guidance information as a result of the triglyceride level measurement from the wearable device 900, and may output the information to a display 1010. In another example, bio-impedance may be measured by the impedance sensor 910 of the wearable device 900, and the mobile device 1000 receives the bio-impedance data from the wearable device 900 and may measure the triglyceride level based on the received bio-impedance data and user metadata and output a measurement result. The opposite case is also possible.

The present disclosure can be realized as a computer-readable code written on a computer-readable recording medium. The computer-readable recording medium may be any type of recording device in which data is stored in a computer-readable manner.

Examples of the computer-readable recording medium include a ROM, a RAM, a CD-ROM, a magnetic tape, a floppy disc, an optical data storage, and a carrier wave (e.g., data transmission through the Internet). The computer-readable recording medium can be distributed over a plurality of computer systems connected to a network so that a computer-readable code is written thereto and executed therefrom in a decentralized manner. Functional programs, codes, and code segments needed for realizing the present invention can be readily deduced by programmers of ordinary skill in the art to which the invention pertains.

The present disclosure has been described herein with regard to preferred embodiments. However, it will be obvious to those skilled in the art that various changes and modifications can be made without changing technical conception and essential features of the present disclosure. Thus, it is clear that the above-described embodiments are illustrative in all aspects and are not intended to limit the present disclosure.

What is claimed is:

1. An apparatus for measuring blood components, the apparatus comprising:
an impedance sensor configured to measure a bio-impedance of a user; and
a processor configured to measure, by using a multiple-output artificial neural network (ANN) learning model, a concentration of a basic blood component and a concentration of at least one auxiliary blood component associated with the basic blood component, based on user metadata and the measured bio-impedance.

2. The apparatus of claim 1, wherein the multiple-output ANN learning model is pre-trained to output the concentration of the basic blood component and the concentration of the at least one auxiliary blood component.

3. The apparatus of claim 2, wherein the processor further is configured to determine, as the at least one auxiliary blood component, at least one blood component that has a correlation with the basic blood component that is greater than or equal to a threshold value.

4. The apparatus of claim 3, wherein the processor is further configured to, when outputting the basic blood component and the at least one blood component, guide remeasurement based on the correlation between the basic blood component and the at least one auxiliary blood component falling outside a correlation range.

5. The apparatus of claim 3, wherein the basic blood component comprises triglyceride, and the at least one auxiliary blood component comprises uric acid and creatinine.

6. The apparatus of claim 1, wherein the processor is further configured to obtain the user metadata via a user interface, and
wherein the user metadata comprises age, gender, height, and weight.

7. The apparatus of claim 1, wherein the processor is further configured to, based on the measured concentration of the basic blood component and the measured concentration of the at least one auxiliary blood component, provide the user with health guidance, and
wherein the health guidance comprises at least one of a warning, diet recommendations, or exercise information.

8. The apparatus of claim 1, wherein the multiple-output ANN learning model comprises an input layer, a plurality of hidden layers, and an output layer, and
wherein the plurality of hidden layers comprise at least one of a linear function, a batch normalization function, a rectified linear unit function, and a dropout function.

9. The apparatus of claim 1, wherein the processor is further configured to perform preprocessing on the measured bio-impedance and the user metadata by standard scaling.

10. The apparatus of claim 1, wherein the processor is further configured to obtain an impedance index based on the measured bio-impedance and the user metadata, and input the impedance index into the multiple-output ANN learning model.

11. The apparatus of claim 10, wherein the impedance index comprises a value obtained by dividing the user metadata by the measured bio-impedance.

12. A method of measuring blood components by an apparatus for measuring blood components, the method comprising:
measuring, by an impedance sensor, a bio-impedance of a user;
obtaining user metadata from the user through a user interface, the user metadata comprising at least one of age, gender, height, and weight; and
measuring, by using a multiple-output artificial neural network (ANN) learning model, a concentration of a basic blood component and a concentration of at least one auxiliary blood component associated with the basic blood component, based on the measured bio-impedance and the user metadata.

13. The method of claim 12, wherein the multiple-output ANN learning model is pre-trained to output the concentration of the basic blood component and the concentration of the at least one auxiliary blood component.

14. The method of claim 13, wherein the at least one auxiliary blood component comprises a blood component having a correlation with the basic blood component that is greater than or equal to a predetermined threshold value.

15. The method of claim 12, further comprising, based on the measured concentration of the basic blood component and the measured concentration of the at least one auxiliary blood component, providing the user with health guidance comprising at least one of a warning, diet recommendations, and exercise information.

16. The method of claim 12, further comprising performing preprocessing on the measured bio-impedance and the user metadata by standard scaling.

17. The method of claim 12, wherein the measuring the concentration of the basic blood component and the concentration of the at least one auxiliary blood component comprises:
generating an impedance index based on the measured bio-impedance and the user metadata, and
inputting the impedance index into the multiple-output ANN learning model.

18. An electronic device comprising:
a memory storing one or more instructions; and
a processor configured to execute the one or more instructions to:
input a bio-impedance of a user and user metadata into a multiple-output artificial neural network (ANN) learning model, and
output a concentration of a basic blood component and a concentration of at least one auxiliary blood component associated with the basic blood component.

19. The electronic device of claim 18, wherein the multiple-output ANN learning model is pre-trained to output the concentration of the basic blood component and the concentration of the at least one auxiliary blood component.

20. The electronic device of claim 18, wherein the processor is further configured to execute the one or more instructions determine, as the at least one auxiliary blood component, at least one blood component having a correlation with the basic blood component that is greater than or equal to a threshold value.
